(19) United States
(12) Patent Application Publication
(10) Pub. No.: US 2026/0325795 A1
(43) Pub. Date: Oct. 22, 2026

(54) SYSTEMS AND METHODS FOR IDENTIFICATION, SEPARATION, TRACKING, AND MANAGEMENT OF MULTIPLE IV LINES AND ASSOCIATED ACCESS CONNECTORS

(71) Applicant: CleanSite Medical, Inc., Solana Beach, CA (US)

(72) Inventors: John GRANT, Solana Beach, CA (US); Daniel M. CHAMBERS, Solana Beach, CA (US)

(73) Assignee: CleanSite Medical, Inc., Solana Beach, CA (US)

(21) Appl. No.: 19/655,505

(22) Filed: Apr. 22, 2026

Related U.S. Application Data
(60) Provisional application No. 63/792,868, filed on Apr. 22, 2025.

(51) Int. Cl. A61M 25/02; A61M 5/14

(52) U.S. Cl. CPC A61M 25/02; A61M 5/1413; A61M 2025/028; A61M 2205/14; A61M 2205/583; A61M 2205/6063; A61M 2209/082

(57) ABSTRACT

Systems are described that allow a healthcare practitioner, for example, a nurse, to easily distinguish each of a plurality of IV lines connected to a patient, and preferably any connector or fluid access port or the like integrated with or otherwise attached to or included as part of any particular IV line, from any other IV line(s) and/or connectors, ports, and the like that may also be connected to that patient. Methods of using such systems are also described.

### RELATED APPLICATION

This patent application claims the benefit and priority to U.S. provisional patent application serial number 63/792,868, filed on 22 Apr. 2025. The aforementioned priority application is hereby incorporated by reference in its entirety for any and all purposes.

### TECHNICAL FIELD

The invention relates to medical devices useful in treating patients receiving IV therapy, particularly those receiving IV therapy through multiple IV lines.

### Background

In patients receiving therapy through multiple IV lines, healthcare personnel must often rapidly identify a specific IV line and a specific access connector among numerous visually similar lines and connectors located near the patient bedside. Failure to accurately distinguish one IV line from another can result in wrong-line medication administration, including delivery of medication through an unintended lumen, interruption of critical infusions, administration of incompatible drugs through the same line, or accidental flushing of a line intended for a different therapy. Such errors can result in serious patient harm, including hemodynamic instability, medication incompatibility events, and delayed treatment.

Connector confusion presents an additional and distinct clinical problem. A single IV line frequently includes multiple needlefree connectors, Y-sites, stopcocks, valves, extension set connectors, or other fluid access ports positioned at different locations along the line. Healthcare personnel may be unable to determine which connector corresponds to a particular therapy pathway or whether a specific connector was previously accessed. This uncertainty increases the likelihood of accessing the wrong connector, duplicative manipulation of connectors, unnecessary line breaks, and contamination risk.

Missed disinfection events are also a significant source of hospital-acquired infection risk. Before accessing a needlefree connector, standard clinical protocols typically require disinfection of the connector surface using alcohol, disinfecting caps, or other approved approaches. In busy clinical environments, providers may be unable to determine whether a specific connector was recently disinfected, whether a disinfecting cap was removed, or whether another provider previously accessed that connector. Failure to verify connector-specific disinfection can increase the risk of contamination and subsequent bloodstream infection.

Conventional IV line management systems also provide little or no reliable documentation of connector-specific access history. Hospitals frequently cannot determine when a specific connector was last accessed, who accessed it, whether required disinfection occurred, or whether the connector should be replaced. The inability to document access history impairs both bedside decision-making and retrospective review following adverse events.

Similarly, providers often lack a reliable mechanism for determining connector replacement intervals. Many institutional protocols require replacement of connectors, caps, extension sets, or related access components after specified periods of use or after a defined number of access events. Without reliable identification and tracking of individual connectors, replacement decisions may be delayed, inconsistent, or based on estimates rather than verified usage history.

Hospitals also face substantial pressure to reduce central line-associated bloodstream infections (CLABSIs) and other catheter-related infections. Regulatory agencies, accreditation bodies, payors, and internal infection-prevention programs increasingly monitor compliance with disinfection protocols, connector replacement schedules, and line-access documentation. Failure to maintain compliance may increase patient risk, reduce quality metrics, and create reimbursement and liability exposure.

In addition, hospitals increasingly require compliance monitoring and audit capability for IV access workflows. Existing IV line organizers may physically separate lines but do not provide deterministic identification of individual connectors associated with specific IV lines, nor do they support connector-specific access tracking, disinfection verification, audit trail generation, or compliance reporting. Accordingly, there remains a need for systems that not only organize IV lines physically, but also enable reliable identification, tracking, and management of each individual IV line and associated connector throughout patient treatment.

### Summary of the Invention

The object of the invention is a system to allow a healthcare practitioner, for example, a nurse, to easily distinguish each of a plurality needlefree connectors present on one or more IV lines connected to a patient as part of an IV line system, from any other IV line(s) and/or connectors, ports, and the like that may also be part of the IV line system connected to that patient. This is accomplished by providing a hierarchical identification architecture in which each IV line in an IV line system connected to a patient has a unique line identifier, each connector (or port or other fluid access point) in or on a particular IV line has a unique connector identifier. This allows each connector in the IV line system to be identified and managed through a combination of the IV line identifier and the connector identifier. If desired, the systems of the invention can also include an IV separator to provide physical positional mapping to assist in such identification and management.

Thus, an aspect of the invention concerns systems of components that allow nurses, for example, to readily uniquely identify and manage a plurality of needlefree connectors in an IV line system connected to a patient. Such systems typically include for each IV line in the IV line system, at least one attachable distinguishing IV line label for that IV line, and wherein when a plurality of IV lines is present in the IV line system, each attachable distinguishing IV line label is distinguishable from any other distinguishing IV line label attached to an IV line connected to the patient; and for each needlefree connector in the IV line system, a unique attachable distinguishing connector label that allows each needlefree connector on the IV line of which such needlefree connector is a part to be distinguished from any other needlefree connector(s) on that IV line. This allows any needlefree connector on any IV line in the IV line system to be distinguished from any other needlefree connector(s) in the IV line system connected to the patient.

In many preferred embodiments, the system further includes a third component, namely an IV line separator configured to removably separate and retain a plurality of IV lines attached to a patient. Advantageously, in such a system the IV line separator comprises a plurality of independently addressable IV line channels each configured to removably separate and retain IV tubing without restricting fluid flow though IV tubing when disposed in the IV line separator. Advantageously, in such a system the IV line separator may further comprise an attachment element configured to removably attach the IV line separator to a patient bed, optionally a handrail, or an IV pole. Each independently addressable IV line channel is advantageously visibly labelled with a unique IV line identification symbol, optionally an alphanumeric character.

Each attachable distinguishing IV line label is advantageously visibly labelled with a unique IV line identification symbol, optionally an alphanumeric character, and wherein each attachable distinguishing IV line label optionally is the marked with the unique IV line identification symbol for that IV line.

Each attachable distinguishing connector label is advantageously visibly independently labelled with a connector identification symbol, optionally an alphanumeric character, and wherein each attachable distinguishing connector label optionally is the marked with the unique connector identification symbol for the needlefree connectors in that IV line.

Another aspect of the invention concerns methods of method of tracking a plurality of IV lines connected to a patient. Such methods typically comprising using a system according to claim 1 to track use of each of the IV lines in the plurality of IV lines. Advantageously, such methods can further comprise tracking use of any needlefree connector(s) included in the plurality of IV lines. Such tracking advantageously employs software configured to store and analyze data corresponding to use of the IV line separator, IV lines, and needlefree connectors used to treat the patient.

A related aspect of the invention concerns methods of using a system according to the invention to track when such a system is placed on a patient and when an IV line connected to the patient, or a needlefree connector included in one of the IV lines connected to the patient, is used.

### BRIEF DESCRIPTION OF THE DRAWINGS

These and other aspects will now be described in detail with reference to the following drawings. Like reference symbols in the various drawings indicate like elements.

FIG. 1 shows two drawings, A and B, of conventional IV line organizers (1, 2). The drawing in FIG. 1A shows four gravity flow IV lines (3-6). The drawing in FIG. 1B shows eight infusion pump driven IV lines (7-14), with two different IV line organizers (2a, 2b) each holding four different IV lines.

FIG. 2 shows a card (20) to which is attached 5 sets (21-25) of 4 IV line labels (26a-e, 27a-e, 28a-e, 29a-e).

FIG. 3 shows a card (50) to which is attached 5 sets (60-64) of 6 attachable distinguishing IV connector labels (A-F).

The details of one or more embodiments are set forth in the accompanying drawings and the description below. Other features and advantages will be apparent from the description and drawings, and from the claims.

### DETAILED DESCRIPTION

The specification, the attached drawings, and the appended claims describe articles, namely systems and methods, to allow healthcare practitioners, e.g., nurses, to easily distinguish each of needlefree connectors on one or a plurality of IV lines connected to a patient as part of an IV line system from any other IV line(s) and/or connectors, ports, and the like that may also be connected to that patient.

As is known, acutely ill, hospitalized patients with life-threatening conditions require constant monitoring and treatment, which often requires the administration of several different medications at the same time. Many such medications are administered directly into patients’ veins (i.e., intravenously), often using programmable infusion pumps to control the rate and duration of medication delivery. When more than one medication is administered this way (so-called multiple IV infusions) to a patient, mistakes can happen that can pose serious, even life-threatening, risks to the patient. Some of these mistakes result from tangled IV lines, which can cause the misidentification of IV lines and the needlefree connectors included in such lines.

To reduce the risks of such mistakes, this invention provides systems of components that allow nurses, for example, to readily distinguish one needlefree connector from any other connector that is part of an IV line system connected to a patient. Here, an IV line system refers to one or more IV lines, and the one or more connectors, ports, or IV access points in or on and of the IV lines connected to a patient. The systems of the invention typically include at least one attachable distinguishing IV line label for each of any one or more IV lines in an IV line system connected to a patient and at least one attachable distinguishing connector label for each needlefree connector present in the IV line system connected to the patient in order to allow a first needlefree connector on the first IV line to be distinguished from any other needlefree connector(s) on the first IV line and, in combination with an attachable distinguishing IV line label attached to the first IV line, to allow the first needlefree connector on the first IV line to be distinguished from any other needlefree connector(s) on other IV lines connected to the patient, and in many embodiments the system further includes at least one additional component, namely an IV line separator configured to removably separate and retain a plurality of IV lines attached to a patient. Other components may, of course, also be included, such as those that identify when an IV line is established when it was last changed, the name of the drug(s) (or other therapy(ies)) being administered through the particular IV line, etc.

In preferred embodiments, each needlefree connector or other fluid access port in an IV line system connected to a patient is uniquely identifiable by a hierarchical identifier composed of (i) an IV line identifier associated with the IV line to which the connector belongs and (ii) a connector-specific identifier unique among connectors associated with that IV line. For example, a connector may be identified as Line 2–Connector C. Such a relational identification structure enables deterministic identification of every connector in an IV system connected to a patient and supports accurate recording of connector-specific access, flushing, disinfection, replacement, and other therapeutic or connector/line management events.

Each attachable distinguishing IV line label is advantageously visibly labelled with a unique IV line identification symbol, optionally an alphanumeric character, and wherein each attachable distinguishing IV line label optionally is the marked with the unique IV line identification symbol for that IV line.

Each attachable distinguishing connector label is advantageously visibly independently labelled with a connector identification symbol, optionally an alphanumeric character, and wherein each attachable distinguishing connector label optionally is the marked with the unique connector identification symbol for the needlefree connectors in that IV line.

In representative embodiments, the distinguishing IV line labels and connector labels useful in the context of the invention are adhesive labels configured for secure but removable attachment to IV tubing, needlefree connectors, Y-sites, stopcocks, valves, extension sets, disinfecting caps, or other access components associated with IV therapy. They can be presented at the bedside for easy healthcare practitioner use on cards, strips, or any suitable format. In some embodiments, they may be printed or other made or printed on site, as needed.

The adhesive labels may be formed from flexible polymeric films, laminated paper materials, synthetic paper, polypropylene, polyethylene, polyester, vinyl, Tyvek®, or other medical-grade label substrates suitable for use in hospital environments. Preferably, the labels are moisture resistant, alcohol resistant, tear resistant, and capable of maintaining legibility during routine patient care, cleaning, and handling.

In some embodiments, the adhesive labels include a pressure-sensitive adhesive configured to adhere securely to polymeric IV tubing, connector housings, or disinfecting caps without interfering with fluid flow, connector operation, sterility, or access to functional surfaces of the connector. The adhesive may be selected to permit removal and repositioning without leaving substantial residue, damaging the IV line, or obscuring manufacturer information already present on the device.

In certain embodiments, the labels are wrap-around labels configured to partially or fully encircle IV tubing. In other embodiments, the labels are flag-style labels that extend outwardly from the tubing to improve visibility while minimizing obstruction of adjacent components. In still other embodiments, the labels comprise small-format labels sized for placement directly on or adjacent to a needlefree connector body, extension set housing, stopcock handle, disinfecting cap, or other fluid access port.

The labels may include a pull tab, release tab, liner tab, or non-adhesive handling region that facilitates sterile or gloved application by a healthcare practitioner. Labels may be supplied on a release liner, card, sheet, roll, cartridge, dispenser, or sterile packaging system. In preferred embodiments, multiple coordinated sets of labels are provided on a single carrier card such that all labels associated with a particular patient or IV line can be selected together.

Visible indicia on the labels may include alphanumeric characters, colors, symbols, geometric shapes, arrows, treatment-specific markings, barcodes, QR codes, RFID elements, NFC elements, machine-readable identifiers, or combinations thereof. In preferred embodiments, each IV line label carries a unique IV line identifier and each connector label carries a connector-specific identifier associated with a particular IV line, thereby creating a hierarchical identification structure.

In some embodiments, the labels further include writable regions for manual notation by healthcare personnel, including medication names, placement dates, replacement dates, access times, initials of the provider, or disinfection status. In other embodiments, the labels are preprinted and sterile, and are intended for single-patient disposable use.

The size, shape, adhesive properties, and placement of the labels may vary depending on the type of connector or IV tubing to which the label is applied, and the invention is not limited to any particular label geometry or attachment mechanism, provided that the labels permit reliable identification of individual IV lines and associated connectors during patient care.

In addition to adhesive labels, the systems described herein may employ a variety of alternative attachment and identification structures for distinguishing IV lines and associated connectors.

In some embodiments, the identifiers comprise clip-on labels configured to releasably attach to IV tubing, connector housings, extension sets, Y-sites, stopcocks, disinfecting caps, or other fluid access components. Such clip-on labels may include resilient arms, hinged members, spring features, or deformable retention elements that allow the identifier to be manually attached and removed without damaging the underlying device. Clip-on labels may be particularly useful where repeated repositioning is desired or where adhesive attachment is less preferred.

In certain embodiments, the identifiers comprise snap-fit labels configured to engage a connector body or tubing segment through a mechanical interference fit. For example, a snap-fit label may include a semi-cylindrical or annular body that snaps over a needlefree connector housing or a tubing segment and remains securely retained during use. Snap-fit labels may provide improved durability and resistance to accidental removal during high-activity clinical workflows.

In some embodiments, the identifier is provided as a molded label or molded identifier formed as a discrete polymeric component carrying visible indicia such as numbers, letters, symbols, colors, or machine-readable markings. Such molded labels may be separately attached to an IV line or connector by adhesive, clip, snap-fit engagement, or other mechanical retention methods.

In other embodiments, connectors useful in the context of the invention are labeled during manufacture, advantageously during IV set assembly, with a distinguishing connector label. For example, a portion of an exterior surface of a connector can be labeled with distinguishing connector label that allows that connector to be distinguished from any other connector in the particular IV set. In this way, such an IV set comes with its connectors pre-labeled, each distinguishably labeled from the other(s) in the IV set.

In other embodiments, the identifying structure is integrated directly into the molded connector itself. For example, a needlefree connector, stopcock, valve, extension set component, disinfecting cap, or other access device may be manufactured with a permanently integrated molded identifier, such as a raised number, recessed marking, molded color region, molded symbol, embedded machine-readable feature, or structural feature that provides unique identification without requiring a separate applied label. Integrated molded identifiers may improve manufacturing consistency and reduce the risk of identifier loss during use.

In some embodiments, the identifier comprises a heat-shrink label configured to be positioned around IV tubing or a connector component and subsequently shrunk into secure engagement through the application of heat. Heat-shrink labels may provide low-profile identification with high durability and resistance to moisture, cleaning agents, and routine handling. Such labels may be transparent, opaque, color-coded, or include printed or machine-readable indicia.

In certain embodiments, the identifiers are sterile labels provided in sterile packaging for use during line setup, sterile field procedures, or sterile connector replacement workflows. Sterile labels may be individually packaged, supplied in sterile trays, or incorporated into sterile procedure kits to support aseptic handling and application by healthcare personnel.

In some embodiments, the identifiers are intended for single-patient disposable use and comprise disposable labels configured to remain with the IV line system during a patient treatment period and be discarded thereafter. Disposable labels may be particularly useful for infection-prevention workflows and hospital compliance protocols requiring replacement between patients.

In other embodiments, the identifiers comprise reusable labels configured for repeated sterilization, disinfection, or cleaning between uses. Reusable labels may be formed from autoclavable polymers, disinfectant-resistant materials, or other durable substrates capable of repeated hospital use while maintaining legibility and structural integrity. Reusable identifiers may be especially useful for reusable separator systems or long-term line management applications.

The invention is not limited to any particular identifier attachment mechanism, construction, or material, and adhesive labels, clip-on labels, snap-fit labels, molded identifiers, integrated molded connector identifiers, heat-shrink labels, sterile labels, disposable labels, reusable labels, and combinations thereof may be used alone or in coordinated systems to provide reliable identification of IV lines and associated connectors.

The identifiers used for distinguishing IV lines and associated connectors may employ a wide variety of visible, machine-readable, or electronically detectable identification modes, and the invention is not limited to any particular marking format.

In some embodiments, the identifiers comprise alphanumeric markings, including letters, numbers, combinations of letters and numbers, serial identifiers, sequence identifiers, or hierarchical identifiers. For example, an IV line may be designated by a numeric identifier such as “Line 2,” while individual connectors associated with that IV line may be designated by alphabetic identifiers such as “A,” “B,” and “C,” such that a specific connector is identified as “Line 2–Connector C.” Alphanumeric identifiers may be printed, molded, embossed, engraved, written, or otherwise visibly displayed.

In certain embodiments, identification is achieved using color coding. Different IV lines may be assigned different colors, and connectors associated with a particular line may use coordinated or derivative color schemes to reinforce visual association. Color coding may be used alone or in combination with alphanumeric identifiers to improve rapid recognition in high-acuity clinical settings. Colors may be applied through printed labels, molded polymer components, colored sleeves, coatings, inks, or integrated molded connector features.

In some embodiments, the identifiers comprise symbols, including geometric shapes, icons, arrows, directional indicators, treatment-specific symbols, warning symbols, or other distinguishable visual markers. Symbols may be especially useful where language neutrality, rapid recognition, or reduced reading burden is desired. Symbols may be used independently or in combination with colors and alphanumeric identifiers.

In certain embodiments, the identifiers comprise barcodes configured to store machine-readable information associated with an IV line or connector. Barcodes may be linear barcodes, stacked barcodes, or other machine-readable formats capable of being scanned by hospital barcode readers, mobile devices, infusion systems, or bedside documentation systems. Barcode data may correspond to line placement, access history, replacement schedules, medication assignment, or patient-specific workflow tracking.

In some embodiments, the identifiers comprise QR codes or other two-dimensional machine-readable codes capable of storing expanded information relative to conventional linear barcodes. QR codes may encode connector identity, IV line identity, placement time, disinfection history, replacement intervals, patient identifiers, medication assignments, or links to electronic medical record (EMR) systems. QR code scanning may support rapid bedside verification and audit trail generation.

In certain embodiments, the identifiers comprise radio-frequency identification (RFID) elements, including passive RFID tags, active RFID tags, or semi-passive RFID devices associated with IV lines, connectors, disinfecting caps, or separator components. RFID systems may permit non-line-of-sight identification, automated inventory tracking, replacement monitoring, and workflow documentation without requiring direct visual scanning of the identifier.

In some embodiments, the identifiers comprise near-field communication (NFC) elements configured to permit close-range electronic interrogation using dedicated readers, mobile devices, infusion pumps, or hospital workflow systems. NFC identifiers may be particularly useful for secure verification of access events, disinfection confirmation, provider documentation, and integration with electronic charting systems.

Multiple identification modes may be used together in coordinated systems. For example, a connector may include a visible alphanumeric identifier for bedside recognition, a color-coded visual marker for rapid workflow identification, and a machine-readable QR code or RFID element for electronic tracking and compliance monitoring. Such combined identification modes may improve both human usability and system-level documentation.

The invention is not limited to alphanumeric identifiers, color coding, symbols, barcodes, QR codes, RFID elements, NFC elements, or any specific identifier technology, and any now known or later developed identification mode capable of reliably distinguishing IV lines and associated connectors may be employed.

Advantageously, in systems of the invention that include at least one IV line separator, that component comprises a plurality of independently addressable IV line channels each configured to removably separate and retain IV tubing without restricting fluid flow though IV tubing when disposed in the IV line separator. Advantageously, in such a system the IV line separator may further comprise an attachment element configured to removably attach the IV line separator to a patient bed, optionally a handrail, or an IV pole. Each independently addressable IV line channel is also advantageously visibly labelled with a unique IV line identification symbol, optionally an alphanumeric character.

The IV line separator may be configured for temporary or semi-permanent attachment at a variety of locations near the patient in order to support organized line management during treatment. The invention is not limited to any particular mounting structure or placement location.

In some embodiments, the IV line separator comprises a bed rail mount configured to removably attach the separator to a patient bed rail, handrail, side rail, transport rail, stretcher rail, or other structural member adjacent the patient. The bed rail mount may include hooks, clips, brackets, snap-fit members, flexible straps, hook-and-loop fasteners, releasable latches, or other retention structures suitable for rapid bedside installation and removal. Bed rail mounting may be particularly advantageous because it places the separator in close proximity to the patient access site and allows healthcare personnel to quickly trace lines from the patient to their associated connectors.

In certain embodiments, the IV line separator comprises an IV pole mount configured for attachment to a conventional IV pole, infusion stand, rolling pole assembly, or similar upright support structure. The IV pole mount may include clamps, collars, straps, snap-fit sleeves, hook structures, threaded fasteners, or adjustable retention members capable of accommodating IV poles of different diameters. IV pole mounting may be useful where line organization is desired near infusion pumps, fluid sources, or medication administration equipment located away from the patient bed.

In some embodiments, the separator comprises an infusion pump mount configured to attach directly to an infusion pump housing, pump pole bracket, pump docking station, or associated support structure. Such mounting may allow direct correlation between a specific infusion pump channel and a corresponding IV line retained within the separator, thereby improving medication administration accuracy and reducing pump-to-line confusion.

In certain embodiments, the separator comprises an adhesive mount configured to temporarily adhere the separator to a patient bed, bedside table, pump housing, monitor stand, wall surface, procedure cart, or other nearby structure. The adhesive mount may include pressure-sensitive adhesive pads, removable adhesive strips, medical-grade adhesive surfaces, double-sided adhesive materials, or repositionable adhesive systems. Preferably, the adhesive mount permits secure retention during normal patient care while allowing removal without substantial residue or damage to the mounting surface.

In some embodiments, the separator comprises a clamp mount configured to mechanically engage a bed rail, IV pole, pump housing, procedure cart, or similar support structure. Clamp mounts may include spring clamps, screw clamps, hinged clamps, cam-lock clamps, friction clamps, or other adjustable gripping structures capable of secure attachment to support members of varying sizes and geometries. Clamp mounting may provide increased stability in high-movement environments such as emergency departments, intensive care units, or patient transport settings.

The IV line separator may be configured as a disposable separator intended for single-patient use. Disposable separators may be formed from molded plastic, thermoformed polymer, laminated paperboard, synthetic paper, foam structures, or other cost-effective materials suitable for short-term clinical use and subsequent disposal after patient discharge or line replacement. Disposable separators may be particularly advantageous in infection-prevention workflows where cross-patient contamination risk is minimized by single-patient use.

In other embodiments, the IV line separator comprises a reusable separator configured for repeated use across multiple patients following cleaning, sterilization, or disinfection. Reusable separators may be formed from durable polymers, autoclavable materials, disinfectant-resistant materials, stainless steel, coated metals, or other robust materials capable of maintaining structural integrity and label visibility after repeated hospital cleaning cycles. Reusable separators may be preferred in high-volume facilities seeking durable long-term line management systems.

In certain embodiments, the separator is provided as a sterile packaged separator supplied in sterile wrapping, peel pouches, sterile trays, procedure kits, or integrated sterile line setup systems. Sterile packaged separators may be especially useful for operating rooms, central line placement procedures, sterile bedside insertions, catheter replacement procedures, and other workflows where maintenance of sterility is clinically important. Sterile packaged embodiments may be disposable or reusable depending on the intended application.

The invention is not limited to bed rail mounts, IV pole mounts, infusion pump mounts, adhesive mounts, clamp mounts, disposable separators, reusable separators, sterile packaged separators, or any specific mounting or construction approach, and multiple mounting features may be combined in a single separator system depending on the intended clinical environment.

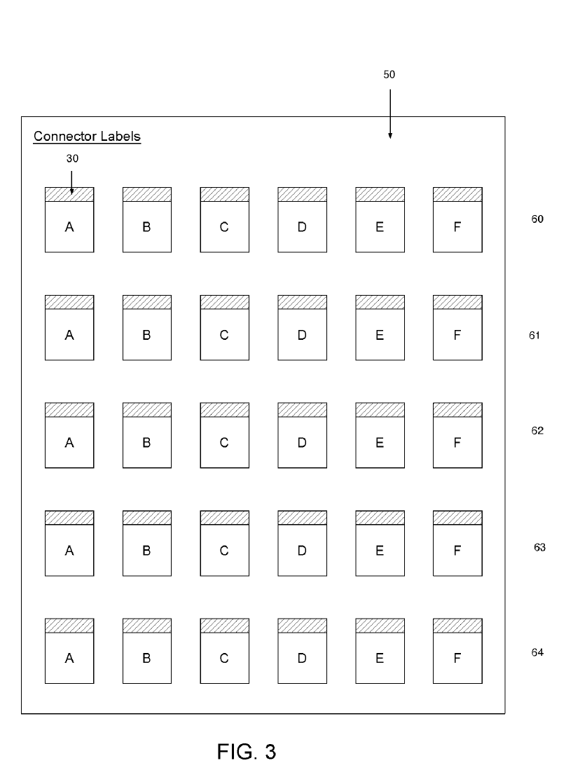

As will be appreciated, markings of the various components of the systems of the invention can be, for example, alphanumeric (as shown, for example, in FIG. 3). Of course, any other now known or later developed distinguishing characters or labels (or other approaches for product or inventory tracking) can also be used, for example, QR codes, UBC codes, colors, and the like. In this way, each IV line and each connector in an IV line attached to a patient can be readily distinguished from any other line and/or connector, and any other connector in any other IV line, attached to the patient. If all connectors and IV lines attached to a patient are so labeled, each connector on each line can be readily distinguished from the other connectors on that line, as well as from other connectors on other IV lines (when the IV line labels for those IV lines are also considered). This will allow tracking of the use of any, some, or all of the connectors present on any IV line attached to a patient, even if multiple IV lines each with two or more connectors is attached to a patient.

Thus, another aspect of the invention concerns methods of method of tracking a plurality of needlefree connectors and IV lines connected to a patient. Such methods typically include using a system of the invention to track use of and, advantageously, reduce or prevent entanglement of, each of the IV lines in the plurality of IV lines. Advantageously, such methods can further include tracking use of any needlefree connector(s) included in the plurality of IV lines. Such tracking advantageously employs software configured to store and analyze data corresponding to use of IV line separators, IV lines, and needlefree connectors used to treat the patient.

A related aspect of the invention concerns methods of using a system according to the invention to track when such a system is placed on a patient and when an IV line connected to the patient, or a needlefree connector included in one of the IV lines connected to the patient, is used.

Thus, In certain embodiments, the systems described herein further include software configured to record, store, analyze, and report information associated with the placement, identification, access, disinfection, replacement, and management of IV lines and associated connectors. Such software may operate on a bedside workstation, mobile device, tablet, infusion pump interface, central nursing station, hospital server, cloud-based platform, or other computing system.

In some embodiments, the software is configured for electronic medical record (EMR) integration. Information associated with individual IV lines and connectors, including placement dates, access events, disinfection events, replacement schedules, medication assignments, and provider actions, may be transmitted to and from the patient’s EMR. EMR integration may permit automatic documentation of IV access activity, reduce manual charting burden, improve compliance reporting, and support retrospective review of patient treatment history.

In certain embodiments, the system supports scan event logging in which a healthcare practitioner scans a barcode, QR code, RFID element, NFC element, or other machine-readable identifier associated with an IV line, connector, disinfecting cap, or separator component before or after a clinical action. Scan events may correspond to connector access, medication administration, disinfection confirmation, line replacement, cap replacement, or patient transfer events. Each scan event may be automatically time-stamped and associated with a specific patient, provider, and identified connector. n some embodiments, the software supports nurse workflow documentation by recording routine line-management activities performed during patient care. Such documentation may include initial placement of the separator, assignment of identifiers to IV lines, connector access, medication administration, connector replacement, dressing changes, line tracing verification, and disinfection confirmation. Workflow documentation may reduce duplicate work, improve handoff communication between providers, and support institutional line-management protocols.

In certain embodiments, the software is configured for disinfection logging. Before accessing a connector, a healthcare practitioner may record completion of a disinfection event manually or through a scan event associated with removal of a disinfecting cap, use of an alcohol preparation device, or scanning of the connector itself following disinfection. The system may store the time of disinfection and associate that event with the specific identified connector, thereby allowing subsequent verification that required disinfection occurred prior to access.

In some embodiments, the software generates replacement interval alerts for connectors, disinfecting caps, IV sets, administration sets, extension sets, stopcocks, valves, or other IV access components. Replacement intervals may be based on elapsed time since placement, elapsed time since prior replacement, number of access events, institutional policy rules, manufacturer recommendations, or combinations thereof. When a replacement threshold is reached or exceeded, the system may generate a visual, audible, electronic, or EMR-based alert to prompt timely replacement.

In certain embodiments, the software performs compliance monitoring by evaluating whether required clinical protocols have been followed for identified IV lines and connectors. For example, the system may determine whether connector disinfection occurred within a required time window before access, whether replacement intervals were exceeded, whether required documentation was completed, or whether a provider accessed the correct connector associated with a prescribed therapy. Compliance monitoring may support infection-prevention programs, nursing quality initiatives, and hospital accreditation requirements.

In some embodiments, the system generates an audit trail comprising a chronological record of events associated with each identified IV line and connector. The audit trail may include placement time, provider identity, access events, disinfection events, medication administration events, replacement events, alerts generated, and compliance outcomes. Audit trails may be used for clinical review, adverse event investigation, infection-prevention analysis, reimbursement support, regulatory compliance, quality assurance review, and defense of malpractice or hospital liability claims.

Multiple software functions may be integrated into a unified workflow management platform such that physical identification of IV lines and connectors at the bedside is directly linked to electronic documentation, compliance verification, and institutional reporting. In this manner, the invention improves not only physical line organization but also the reliability, traceability, and safety of IV therapy management throughout patient treatment.

### Description of Representative Embodiments

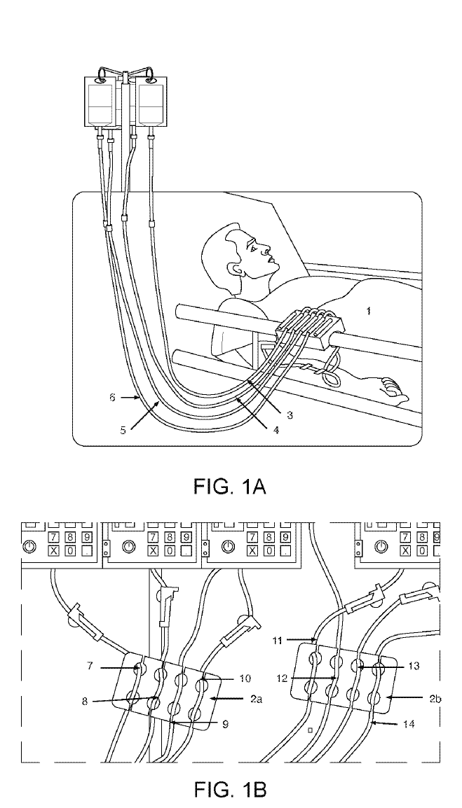

FIG. 1 shows two drawings, A and B, of representative IV line organizers (1, 2) useful in the context of the invention. The drawing in FIG. 1A shows four gravity flow IV lines (3-6) connected to a patient. The drawing in FIG. 1B shows eight infusion pump driven IV lines (7-14) connected to a patient, with two different IV line organizers (2a, 2b) each holding four different IV lines (7-10 and 11-14).

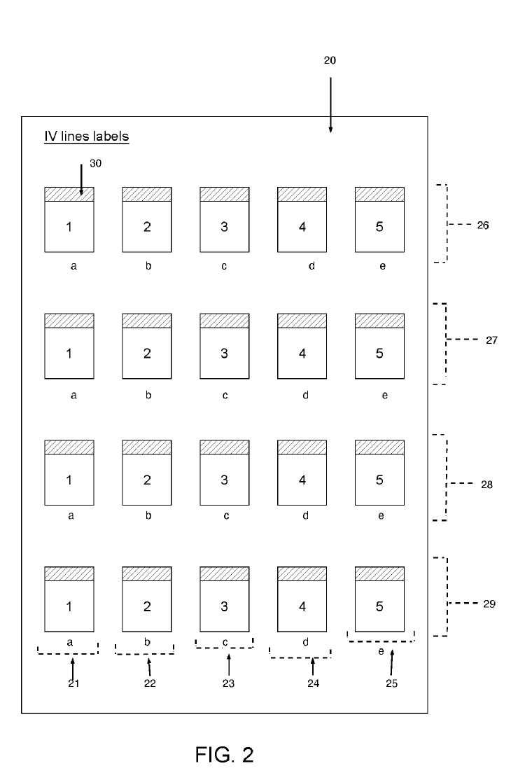

FIG. 2 shows a card (20) to which is attached 5 sets (21-25) of 4 IV line labels (26a-e, 27a-e, 28a-e, 29a-e). In the representative embodiment depicted, each label is peelable from the card and includes a pull tab (shaded area, 30). After peeling the label from the card, it can be attached to a desired IV line. If desired, several labels from the same set of IV line labels can be attached at different locations along the length of a particular IV line.

Each set of IV line labels is numbered, 1, 2, 3, 4, or 5 (also a-e). Within each set of IV line labels, the labels contain the same number. In this way, a nurse can use 1 or more labels within a given set of IV line labels to label a particular IV line along its length, for example near its proximal end (i.e., nearest the patient) and distal end (i.e., nearest the IV bag to which the IV line is attached) ends. Different numbers of sets of IV line labels, each set being distinguishably marked from other sets of IV line labels, can be used, depending on the number of IV lines being used (or contemplated for use) with a particular patient. Markings can be, for example, alphanumeric. Other distinguishing characters or labels (or other approaches for product or inventory tracking) can also be used, for example, QR codes, UBC codes, colors, and the like. In this way, each IV line attached to a patient can be readily distinguished from any other IV line attached to the patient. If all IV lines are attached to a patient are so labeled, each line can be readily distinguished from the others, which facilitates tracking the use and characteristics of such lines and connectors, for example, when the IV line was connected to a patient or last changed, when the particular connector was last accessed, etc.

FIG. 3 shows a card (50) to which is attached 5 sets (60-61) of 6 attachable distinguishing IV connector labels (A-F). In the representative embodiment depicted, each of these attachable distinguishing IV connector labels is peelable from the card and includes a pull tab (shaded area, 30). After peeling the label from the card, it can be attached to, or near, a desired IV connector on an IV line connected to (or to be connected to) a patient. If desired, several labels from the same set of attachable distinguishing IV connector labels can be attached on or near different connectors spaced at different locations along the length of a particular IV line.

Each set of attachable distinguishing IV connector labels is preferably marked in a manner that allows each connector on the IV set of which the particular connector is a part to be assigned a label that enables that connector to be distinguished from every other connector on the same IV line. In the embodiment shown in FIG. 3, within each set (60-64) the attachable distinguishing IV connector labels are marked as A, B, C, D, E, and F. In other words, within each set of attachable distinguishing IV connector labels, the labels are differentially marked so that they can be distinguished from each other, thereby allowing the connectors of a particular IV line to be readily distinguished from each other. In this way, these labels can be used to mark different connectors in an IV line in a way that allows the connectors of that particular IV line to each be distinguished from that IV line’s other connector(s). In this way, a nurse can use 1 or more labels within a given set of attachable distinguishing IV connector labels to distinguishably label each of the connectors along a particular IV line over its length, from the IV line’s proximal to distal ends. The number of IV lines used, or connected to the patient (e.g., 1, 2, 3, 4, 5, 6, 7, 8, 10, 11, 12, 13, 14, 15, 16, 17, 18, 19, 20, or more IV lines), and the number of connectors per line (connector numbers and types can vary between different IV lines), will dictate the number of sets and attachable distinguishing IV connector labels needed to label all of the connectors present on the IV lines for a particular patient such that not only the IV lines, but also each of the connectors present, can be uniquely labeled and identified. In other words, on a patient having multiple IV lines connected where each line has multiple connectors to provide fluid access to the patient, this system allows a specific connector on a specific line to be distinguished from every other connector that is then part of that patient’s multiple IV infusion arrangement. For example, on a patient having 7 IV lines simultaneously connected to deliver different medications, here, lines 1-7, where each IV line has 3 needlefree connectors, a proximal connector (nearest the patient, here identified with an attachable distinguishing connector identifier or label, “A”, from a set of attachable distinguishing connector identifiers or labels A-E), a distal connector (nearest the IV bag, here identified with an attachable distinguishing connector identifier or label, “C”, from the set of attachable distinguishing connector identifiers or labels A-E), and a central connector (positioned between the proximal and distal connectors along the IV line, here identified with an attachable distinguishing connector identifier or label, “B”, from the set of attachable distinguishing connector identifiers or labels A-E), the most proximal connector on IV line 3 (labeled at least near its proximal and distal ends with an attachable distinguishing IV line label “3”) could be labeled, for example, “3A”, meaning IV line 3, connector A. This would allow that connector (3A) to be distinguished from each of the other 20 connectors spread across the patient’s multiple IV infusion arrangement.

Unless the context clearly requires otherwise, throughout the description above and the appended claims, the words "comprise," "comprising," and the like are to be construed in an inclusive sense as opposed to an exclusive or exhaustive sense; that is to say, in a sense of "including, but not limited to." Words using the singular or plural number also include the plural or singular number, respectively. Additionally, the words "herein," "hereunder," "above," "below," and words of similar import refer to this application as a whole and not to any particular portions of this application. When the word "or" is used in reference to a list of two or more items, that word covers all of the following interpretations of the word: any of the items in the list, all of the items in the list, and any combination of the items in the list.

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law

1. A system for uniquely identifying and managing a plurality of needlefree connectors in an intravenous (IV) line system connected to a patient, the system comprising:
a. for each IV line in the IV line system, at least one attachable distinguishing IV line label for that IV line, and wherein when a plurality of IV lines is present in the IV line system, each attachable distinguishing IV line label is distinguishable from any other distinguishing IV line label attached to an IV line connected to the patient;
b. for each needlefree connector in the IV line system, a unique attachable distinguishing connector label that allows each needlefree connector on the IV line of which such needlefree connector is a part to be distinguished from any other needlefree connector(s) on that IV line; and, when the IV line system comprises a plurality of IV lines, optionally
c. an IV line separator configured to removably separate and retain a plurality of the IV lines attached to the patient in the IV line system,
wherein each needlefree connector in the IV line system connected to the patient can be uniquely identified by a combination of a distinguishing IV line label and distinguishing connector label, in an intravenous (IV) line system connected to a patient.

2. A system according to claim 1 comprises an IV line separator, wherein the IV line separator comprises a plurality of independently addressable IV line channels each configured to removably separate and retain IV tubing without restricting fluid flow though the IV tubing.

3. A system according to claim 1 comprises an IV line separator, wherein the IV line separator further comprises an attachment element configured to removably attach the IV line separator to a patient bed, optionally a handrail, or an IV pole.

4. A system according to claim 2 wherein each independently addressable IV line channel is visibly labelled with a unique IV line identification symbol, optionally an alphanumeric character.

5. A system according to claim 2 wherein each attachable distinguishing IV line label is visibly labelled with a unique IV line identification symbol, optionally an alphanumeric character, and wherein each attachable distinguishing IV line label optionally is the marked with the unique IV line identification symbol for that IV line.

6. A system according to claim 2 wherein each attachable distinguishing connector label is visibly independently labelled with a connector identification symbol, optionally an alphanumeric character, and wherein each attachable distinguishing connector label optionally is the marked with the unique connector identification symbol for the needlefree connectors in that IV line.

7. A method of tracking a plurality of IV lines connected to a patient, the method comprising using a system according to claim 1 to track use of each of the IV lines in the plurality of IV lines.

8. A method according to claim 7 that further comprises tracking use of any needlefree connector(s) included in the plurality of IV lines.

9. A method according to claim 7 wherein the tracking employs software configured to store and analyze data corresponding to use of the IV line separator, IV lines, and needlefree connectors used to treat the patient.

10. A method of using a system according to claim 1, comprising tracking when the system is placed on the patient and when an IV line connected to the patient, or a needlefree connector included in one of the IV lines connected to the patient, is used.
